(19) United States
(12) Patent Application Publication
(10) Pub. No.: US 2003/0180348 A1
(43) Pub. Date: Sep. 25, 2003

(54) Transcellular drug delivery system

(76) Inventors: R. Saul Levinson, Chesterfield, MO (US); Thomas C. Riley, Manchester, MO (US)

Correspondence Address: NATH & ASSOCIATES PLLC, Sixth Floor, 1030 15th Street, N.W., Washington, DC, 20005, US

(21) Appl. No.: 10/102,970

(22) Filed: Mar. 22, 2002

(51) Int. Cl. A61K 9/127

(52) U.S. Cl. 424/450

(57) ABSTRACT

This invention relates to a novel transcellular drug delivery system suitable for controlled delivery of a therapeutically active material across various membranes. The transcellular drug delivery system has a bioadhesive unilamellar vesicle defining an amphiphilic exterior and an aqueous interior, wherein a therapeutically active ingredient is contained inside the aqueous interior.

### BACKGROUND OF THE INVENTION

1. Field of the Invention

The present invention relates to novel transcellular drug delivery systems suitable for controlled delivery of therapeutically active materials across various membranes. The transcellular drug delivery system has a bioadhesive unilamellar vesicle defining an amphiphilic or hydrophobic exterior, an aqueous interior, and a therapeutically active ingredient contained in the aqueous interior. One novel aspect of the transcellular drug delivery system is a docking/release feature whereby the unilamellar vesicle mediates transport of therapeuticcally active materials across various membranes. Another novel aspect of the transcellular drug delivery system is that it provides for oral administration of therapeutic actives which historically could be administered via injection only. The present invention further relates to a method of preparing the unilamellar vesicles.

2. Description of the Related Art

Over the years, methods have been developed to achieve efficient pharmacokinetic delivery of therapeutic drugs through specific membranes. In particular, desirable methods relate to directly delivering to the various membranes therapeutic actives through oral, rectal, nasal, parenteral, intravenous, vaginal, ophthalmic, subcutaneous, cutaneous or pulmonary administration. However, known methods of direct delivery such as conventional high internal phase emulsions (“HIPEs”), esophageal and mucosal bioadhesives, and lipophilic, lipophobic and hydrophilic compositions are severely limited by the presence of biological, chemical, and physical barriers of the various membranes. The known methods of direct delivery of therapeutic actives have yet to address the need for site specific absorption and variable diffusion rates keyed to inherent environmental parameters such as pH, enzyme concentration or VanderWaal interactions on the mucosa. Moreover, biologically active agents such as proteins and peptides are particularly vulnerable to chemical, microbial, enzymatic and pH degradation typical with direct delivery and result in lowered absorption and increased degradation of the therapeutic active when administered via direct delivery.

Oral delivery of therapeutic actives to the circulatory system is the preferred route for administration to animals. However, physical barriers such as skin, the environment of the gastrointestinal tract, lipid bi-layers of membranes and other biological surfaces, and various organ membranes prevent practicable clinical application via oral delivery. One explanation for this phenomenon is because most biologically active agents are labile to various enzymes and are generally unable to penetrate the lipid bilayers of cell membranes. Oral delivery is also impeded by chemical barriers such as varying pH in the GI tract and the presence of powerful digestive enzymes in the oral cavity and GI tract.

In this regard, some active protein agents, such as calcitonin and human growth hormone, may not be readily and effectively delivered orally to the intended cellular target without structural modification or degradation.

In their native 3-dimensional state, proteins are generally partially unfolded and possess their lowest free energy. Although no instrumentation exists to measure free energy, free energy can be generally related to surface free energy and is denoted by ΔG which is the change in the Gibbs free energy. Signal peptides or chaperonins can facilitate a native state protein's ability to cross various cellular membranes. Signal peptides and chaperonins accomplish this by reversibly transforming a protein into a transportable conformation and then re-transforming the protein back to its native state subsequent to transport. The signal peptide or chaperonin then separates from the protein or is cleaved from the protein, allowing the protein to fold into its native state. Gething, M-J., Sambrook, J., Nature, 355, 1992, 33-45.

Similar to signal peptides and/or chaperoning, the synthetic chemical compounds of the known methods mediate protein transport by preventing premature folding of the protein into its native state. The synthetic chemical compound reversibly binds to a biologically active therapeutic and then transports the therapeutic across cellular membranes. Once the drug-carrier crosses the membrane, the complex disassociates.

A known oral delivery technique attempts to overcome protein degradation by protecting the therapeutic active with modified excipients or by adding enzyme inhibitors. For example, insulin modified with amphilic polymers is known to reduce insulin degradation by pepsin or chymotryptin enzymes.

Another technique, known as altered chemical entities (“ACEs”), chemically modifies proteins by the covalent addition of polymers. ACEs are composed of water and fat soluble elements and are covalently bound to small polymers at specific sites on the drug molecule to enhance stability and prevent enzymatic degradation. Chemically altering an active sometimes allows enhanced absorption across membranes and increases the half life of an active in vivo but requires specific and costly development of the modified excipients required for each and every therapeutic active contemplated for oral delivery.

U.S. Pat. No. 6,071,538 (“Milstein et al.”) describes an ACE which is a transportable supramolecular drug/carrier complex. Here, a therapeutic active is reversibly and non-covalently bound with a synthetic chemical compound forming a supramolecular complex. The drug/carrier complex is modeled after natural inter- and intra-cellular transport processes.

However, the ACEs only facilitate transport of macromolecular drug/carrier complexes across any particular membrane and are not site specific. Furthermore, they cannot be administered through two separate absorption routes, i.e., a first release event precludes further release events.

One known method which overcomes the limitations of ACEs and avoids alteration of the active with a synthetic chemical compound is the use of a high free energy protective barrier surrounding the therapeutic active in its native low energy state.

Emulsions having a relatively high ratio of water to oil are known in the art as high internal phase emulsions (“HIPEs”) and possess high free energy. HIPEs have been used in various applications such as fuels, agricultural sprays, textile printing, foods, household and industrial cleaning, cosmetics and drugs, and fire extinguishers. HIPEs have also been used in producing polymeric foam-type materials, for example U.S. Pat. No. 3,988,508 (“Lissant”); and U.S. Pat. No. 5,189,070 (“Brownscombe et al.”).

The most significant feature of known HIPEs is that the emulsions typically break down in the gastrointestinal and/or digestive tracts and lose internal phase energy, which causes coalesce of the emulsion into a continuous film on the mucosal membrane.

Certain liposomes overcome the problem of ACEs by forming a protective barrier over an active agent. For example, U.S. Pat. No. 5,089,278 (“Haynes et al.”) discloses a microwave-activated browning composition for coating food product to produce surface browning, including at least one liposome-encapsulated Maillard browning agent. Additionally, drug delivery systems for insulin and heparin, as described in U.S. Pat. No. 4,239,754 (“Patel et al.”), have been also developed.

U.S. Pat. No. 5,622,930 (“Ahl et al.”) expands upon the drug delivery aspects of liposomes and provides for a method of administering to an animal a liposome composition which reduces adverse physiological reactions. Ahl et al. further provides for a process for making unilamellar vesicles having a diameter of 0.2μ to 5.0μ which are formed by freeze-thaw and extrusion techniques.

Another variant of liposomes also used to deliver pharmaceuticals are microspheres which are defined as artificial polymers of mixed amino acids (proteinoids). U.S. Pat. No. 4,925,673 (“Steiner et al. ”) describes drug-containing proteinoid microsphere carriers as well as methods for their preparation and use. These proteinoid microspheres are useful for the delivery of a number of active agents. U.S. Pat. No. 5,733,752 (“Unger et al.”) also discloses negatively charged microspheres made from amphiphilic lipid materials. However, the microspheres of Unger et al. only release the active ingredients when ruptured by temperature variations or ultrasonic waves and only contemplate topical, inhalation, or subcutaneous delivery.

U.S. Pat. No. 5,474,848 (“Wallach”) relates to a method of producing paucilamellar vesicles made of non-phospholipid surfactants wherein the paucilamellar vesicle must have 2-8 lipid bilayers surrounding a central cavity. Wallach also teaches that small unilamellar vesicles (“SUV's”)have a diameter of 0.20μ or smaller and that large unilamellar vesicles (“LUV's”) have a diameter of 1.0μ or larger. On the other hand, Wallach discloses that unilamellar vesicles are not physically durable and are more likely to be subject to enzymatic degradation.

Yet another variant is taught by U.S. Pat. No. 6,201,065 (“Pathak et al.”). Pathak et al. relates to a gel-forming macromer including at least four polymeric blocks, at least two of which are hydrophobic and at least one of which is hydrophilic, also including a crosslinkable group. The macromers are thermosensitive and possess lipophilicity. Still yet another variant is taught by U.S. Pat. No. 6,165,500 (“Cevc”) which relates to a preparation comprising minuscule droplets of fluid provided with membrane-like structures having a diameter of 0.2μ to 10.0μ and consisting of one or more layers of amphilic molecules.

Nevertheless, the drug delivery systems described above and commonly known in the art do not address: (1) the required, toxic amounts of adjuvants or inhibitors needed in the delivery systems; (2) that suitable low molecular weight therapeutics are not available; (3) the poor stability and inadequate shelf life exhibited by the delivery systems; (4) the difficultly in manufacturing the known systems; (5) protection of the therapeutic active; (6) the adverse alteration of the therapeutic active agent; and/or (7) increased allowance and/or promotion of absorption of the therapeutic active.

Moreover, known liposome drug delivery systems possess relatively limited payloads of therapeutic active per liposome. The limited payload is delivered by a rupturing of the liposome in response to in vivo enzymatic, ultrasonic and/or heat changes, thus exposing the therapeutic active to the environmental degradants. It is believed that exposure of therapeutic actives to the harsh in vivo environment may not always be optimal in the case of all types of drugs, such as proteins and peptides. After rupture, the therapeutic active is targeted to release on a site-specific and selective basis. Thus, the described mechanics of known liposome drug delivery systems result in an unpredictable delivery of therapeutic actives and have the unwanted effect of preventing the liposome from carrying the therapeutic active to more one than one single absorption point, as would be the case in systemic, local or regional delivery systems.

The disadvantages of known systems are overcome with the present inventive subject matter. In particular, the formation of a unilamellar vesicle having a docking/release feature wherein an active is released without rupturing the vesicle; a unilamellar vesicle that is not ultrasonic- or thermo-sensitive; and a unilamellar vesicle having a docking/release feature wherein the vesicle is sufficiently durable to resist enzymatic degradation. These features are achieved while providing for the absorption of labile drugs, i.e. peptides and proteins, through a non-invasive mechanism/approach. This action prevents the pharmaceutically active material from being subjected to acidic, alkaline, enzymatic or other degradation in the GI environment; as well as providing for a method of making a unilamellar vesicle having a docking/release feature wherein the unilamellar vesicle is sufficiently durable to resist enzymatic degradation. This thus provides for the absorption of labile drugs, i.e. peptides and proteins, through a non-invasive mechanism/approach, and thereby prevent allowing the pharmaceutically active material to be subjected to acidic, alkaline, enzymatic or other degradation.

The present inventive subject matter also provides for a transcellular drug delivery system for more than one absorption route; and storage stable vesicles having a substantially globular form which repel each other and possess a high free surface energy state; as well as oral administration of therapeutic actives which historically were administered via injection only.

These and other objects of the invention will be apparent for the detailed description and the claims.

### SUMMARY OF THE INVENTION

The present inventive subject matter relates to a storage stable bioadhesive unilamellar vesicle comprising a) an exterior unilamellar film comprising at least one amphiphilic or hydrophobic material; and b) an aqueous interior defined by said exterior unilamellar film, said aqueous interior comprising a therapeutically active ingredient; wherein said vesicle is from about 100 nm to about 100 microns in size and has a neutral charge associated therewith.

Another embodiment of the present inventive subject matter is a storage stable pharmaceutical composition comprising a) a bioadhesive unilamellar vesicle comprising i) an exterior unilamellar film comprising at least one amphiphilic or hydrophobic material; and ii) an aqueous interior defined by said exterior unilamellar film, said aqueous interior comprising a therapeutically active ingredient; wherein said vesicle is from about 100 nm to about 100 microns in size and has a neutral charge associated therewith; and b) a pharmaceutically acceptable carrier.

Yet another embodiment of the present inventive subject matter is a storage stable bioadhesive unilamellar vesicle comprising a) an exterior unilamellar film comprising at least one amphiphilic or hydrophobic material; and b) an aqueous interior defined by said exterior unilamellar film, said aqueous interior comprising a therapeutically active ingredient; wherein said vesicle is from about 100 nm to about 100 microns in size; and wherein the vesicle further comprises an anionic surfactant.

Another embodiment of the present inventive subject matter is a bioadhesive unilamellar vesicle comprising a) an exterior unilamellar film comprising at least one amphiphilic or hydrophobic material; and b) an aqueous interior defined by said exterior unilamellar film, said aqueous interior comprising water and a leukotriene; wherein said vesicle is from about 100 nm to about 100 microns in size and has a neutral charge associated therewith.

Yet another embodiment of the inventive subject matter is a bioadhesive unilamellar vesicle comprising a) an exterior unilamellar film comprising at least one amphiphilic or hydrophobic material; and b) an aqueous interior defined by said exterior unilamellar film, said aqueous interior comprising water and a cytokine; wherein said vesicle is from about 100 nm to about 100 microns in size and has a neutral charge associated therewith.

Another embodiment of the present inventive subject matter is a method of administering a storage stable labile material, which material is commonly administered as an injectable, to a patient in need thereof, comprising the step of orally, rectally, or via the colon administering to a patient comprising a) a bioadhesive unilamellar vesicle comprising i) an exterior unilamellar film comprising at least one amphiphilic or hydrophobic material; and ii) an aqueous interior defined by said exterior unilamellar film, said aqueous interior comprising a therapeutically active ingredient; wherein said vesicle is from about 100 nm to about 100 microns in size and has a neutral charge associated therewith; and b) a pharmaceutically acceptable carrier.

Another embodiment of the present inventive subject matter is a method of systemically delivering a therapeutically active ingredient to a patient in need thereof, comprising the step of administering a storage stable pharmaceutical composition to said patient, said pharmaceutical composition comprising a) a bioadhesive unilamellar vesicle comprising i) an exterior unilamellar film comprising at least one amphiphilic or hydrophobic material; and ii) an aqueous interior defined by said exterior unilamellar film, said aqueous interior comprising a therapeutically active ingredient; wherein said vesicle is from about 100 nm to about 100 microns in size and has a neutral charge associated therewith; and b) a pharmaceutically acceptable carrier; and wherein said vesicle bioadheres to the tissues of the mouth, throat, esophagus, upper gastrointestinal tract, lower gastrointestinal tract, rectum and colon.

Yet another embodiment of the present inventive subject matter is a method of systemically delivering a pharmaceutically active ingredient to a patient in need thereof, comprising the step of administering a storage stable pharmaceutical composition to said patient, said pharmaceutical composition comprising a) a bioadhesive unilamellar vesicle comprising i) an exterior unilamellar film comprising at least one amphiphilic or hydrophobic material; and ii) an aqueous interior defined by said exterior unilamellar film, said aqueous interior comprising a therapeutically active labile ingredient; wherein said vesicle is from about 100 nm to about 100 microns in size and has a neutral charge associated therewith; and b) a pharmaceutically acceptable carrier; wherein said pharmaceutical composition is administered orally or rectally.

### DETAILED DESCRIPTION OF THE INVENTION

As used herein to describe unilamellar vesicles the terms “substantially globular” or “discrete packets” indicate unilamellar vesicles having a rounded shape produced by high shear homogenization.

As used herein with regard to unilamellar vesicles, the term “bioadhesive” refers to the contact between and the adherence of the vesicles to the surface of living tissues.

As used herein with regard to unilamellar vesicles, the term “average diameter” is the value obtained using a particle size analyzer, such as for example, the SediGraph 5100, which is commercially available from Micromeritics (Norcross, Ga.). Alternatively, average diameter can be determined by measuring the diameters of at least 100 unilamellar vesicles in a photograph(s) taken using an optical microscope.

As used herein with regard to unilamellar vesicles, the term “storage stable” references the in vitro physical stability of the vesicle. Specifically, “storage stable” is used to describe the fact that the aqueous interior of the vesicle does not equilibrate with the carrier during storage of the vesicle, thereby resulting in a vesicle which will not leak or otherwise lose its payload, i.e. the amount of therapeutic active contained within the vesicle, through equilibrating with the carrier.

The term “environmental degradation” is used herein with regard to the chemical effects of the biological environment of the body, i.e., acidic, alkaline or enzymatic, and other chemical or physiological reaction or conditions in the environment upon the vesicle and/or the active ingredient contained therein.

The term “oil” is used herein with regard to the continuous phase of the emulsion and the suspension medium described herein to indicate that these media are hydrophobic and therefore immiscible with the hydrophilic phase. This term does not imply that these two phases must consist of or include oils.

The terms “stable” or “stabilized”, as used herein, means that the unilamellar vesicles formed thereby are substantially resistant to degradation.

The term “biocompatible” as used herein, means a lipid or polymer which, when introduced into the tissues of a human patient, will not result in any severe degree of unacceptable toxicity, including allergenic responses and disease states. Preferably the lipids or polymers are inert.

The present invention relates to a novel transcellular drug delivery system suitable for controlled delivery of a therapeutically active material across various membranes. The transcellular drug delivery system has a bioadhesive unilamellar vesicle defining an amphiphilic or hydrophobic exterior and an aqueous interior, wherein a therapeutically active ingredient is contained inside the aqueous interior. The delivery system is unique because it provides for local, systemic or regional delivery, but does not provide for targeted delivery.

Furthermore, the instant delivery system is unique in that is provides a means for orally administering therapeutics which historically could be administered primarily via parenteral means. In this regard, the present inventive delivery system may now facilitate absorption of such a therapeutic in a local, regional or systemic manner, whereas the therapeutic active previously was capable only of targeted absorption due to its non-oral, i.e., injected, administration. In addition to oral administration, the instant delivery system is capable of rectal administration. The instant delivery system may thus be in the form of a suppository, etc.

Typically, the unilamellar vesicles used in this invention have a diameter from about 0.01μ to about 100μ, i.e. the unilamellar vesicle are from about 100 nm to about 100 microns in size. Preferably, said vesicle is from about 2 microns to about 50 microns in size. Although it is known that larger liposomes tend to be more rapidly cleared from an animal's circulation than a smaller liposome, the bioadhesive unilamellar vesicles of the present inventive subject matter provide for vesicles of varied size. Accordingly, some larger particles release an active in the upper GI tract and some smaller vesicles may release the same active in the lower GI tract.

The unilamellar vesicles of the present invention are constructed from biocompatible lipid or polymer materials, and of these, the biocompatible lipids are especially preferred. For the biocompatible lipid materials, amphiphilic or hydrophobic compositions are preferred. Amphilic compositions refers to any composition of matter which has both lipophilic (hydrophobic properties) and hydrophilic properties.

Hydrophilic groups may be charged moieties or other groups having an affinity for water. Natural and synthetic phospholipids are examples of lipids useful in preparing the stabilized microspheres used in the present invention. They contain charged phosphate “head” groups which are hydrophilic, attached to long hydrocarbon tails, which are hydrophobic. This structure allows the phospholipids to achieve a single bilayer (unilamellar) arrangement in which all of the water-insoluble hydrocarbon tails are in contact with one another, leaving the highly charged phosphate head regions free to interact with a polar aqueous environment. It will be appreciated that a series of concentric bilayers are possible, i.e., oligolamellar and multilamellar, and such arrangements are also contemplated to be within the scope of the presently claimed invention.

The most useful stabilizing compounds for preparing the present unilamellar vesicle wall are typically those which have a hydrophobic/hydrophilic character which allows them to form bilayers, and thus unilamellar vesicles, in the presence of a water based medium. Thus, water, saline or some other water based medium, often referred to hereafter as a diluent, may be an aspect of the unilamellar vesicles of the present invention where such bilayer forming compositions are used as the stabilizing compounds.

Preferred amphilic or hydrophobic materials of use according to the presently claimed invention are selected from the group consisting of mineral oil, lipid material, neutral fats, and mixtures and combinations thereof. A particularly preferred lipid according to the presently claimed invention is a phospholipid.

The stability of the resultant unilamellar vesicles of the present invention may be attributable to the non-Newtonian physical properties demonstrated by vesicles provided by high shear processing. Another notable feature of high shear processing is a high free surface energy and an affinity between vesicles.

The stabilized unilamellar vesicles also posses the unique feature of acquiring a neutral charge which is obtained by a high shear processing technique disclosed herein. The neutral charge is unexpected because the vesicles retain affinity, thereby allowing for greater bioavailability of the active ingredient. This is highly unexpected since it has been previously understood that only charged components were capable of forming a stable structure. It is not necessary to employ auxiliary stabilizing additives, although it is optional to do so, and such auxiliary stabilizing agents would be within the skill of one of ordinarily skilled in the art.

It should be recognized that through the addition of stabilizing additives, the neutral charge of the vesicle may be altered. For example, by employing an anionic surfactant, such as soap, the vesicle may be given a negative charge. Such anionic surfactants would be within the skill of one of ordinary skill in the art and include, but are not limited to docusate sodium and sodium lauryl sulfate.

The biocompatible polymers useful as stabilizing compounds for preparing the unilamellar vesicles used in the presently claimed invention can be of either natural, semi-synthetic or synthetic origin.

As used herein, the term polymer denotes a compound comprised of two or more repeating monomeric units, and preferably 10 or more repeating monomeric units.

The term semi-synthetic polymer, as employed herein, denotes a natural polymer that has been chemically modified in some fashion. Exemplary natural polymers suitable for use in the present invention include naturally occurring polysaccharides. Such polysaccharides include, for example, arabinans, fructans, fucans, galactans, galacturonans, glucans, mannans, xylans (such as, for example, inulin), leavan, fucoidan, carrageenan, galatocarolose, pectic acid, pectin, amylose, pullulan, glycogen, amylopectin, cellulose, dextran, pustulan, chitin, agarose, keratan, chondroitan, dermatan, hyaluronic acid, alginic acid, xanthan gum, starch and various other natural homopolymer or heteropolymers such as those containing one or more of the following aldoses, ketoses, acids or amines: erythrose, threose, ribose, arabinose, xylose, lyxose, allose, altrose, glucose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof.

Exemplary semi-synthetic polymers for use according to the presently claimed invention include carboxymethylcellulose, hydroxymethylcellulose, hydroxypropylmethylcellulose, methylcellulose, and methoxycellulose.

Exemplary synthetic polymers suitable for use in the presently claimed invention include polyethylenes (such as, for example, polyethylene glycol, polyoxyethylene, and polyethylene terephthlate), polypropylenes (such as, for example, polypropylene glycol), polyurethanes (such as, for example, polyvinyl alcohol (PVA), polyvinylchloride and polyvinylpyrrolidone), polyamides including nylon, polystyrene, polylactic acids, fluorinated hydrocarbons, fluorinated carbons (such as, for example, polytetrafluoroethylene), and polymethylmethacrylate, and derivatives thereof.

Additional lipids which may be used to prepare the unilamellar vesicles used in the present invention include but are not limited to: fatty acids, lysolipids, phosphatidylcholine with both saturated and unsaturated lipids including, dioleoyphophatidylcholine, dimyristoyl-phosphatidylcholine, dipentadecanoylphosphatidylcholine; dilauroylphosphatidylcholine; dipalmitoyl-phosphatidylcholine (DPPC); distearoylphosphatidylcholine (DSPC); phosphatidylethanolamines such as dioleoylphosphatidylethanolamine and dipalmitoyl-phosphatidylethanolamine (DPPE); phosphatidylserine; phosphatidylglycerol; phosphatidylinositol; sphingolipids such as sphingomyelin; glycolipids such as ganglioside GM1 and GM2; glucolipids; sulfatides; glycosphingolipids; phosphatidic acids such as dipalymitoylphosphatidic acid (DPPA); palmitic acid; stearic acid; arachidonic acid; oleic acid; lipids bearing polymers such as polyethyleneglycol, i.e., PEGylated lipids, chitin, hyaluronic acid or polyvinylpyrrolidone; lipids bearing sulfonated mono-, di-, oligo- or polysaccharides; cholesterol, cholesterol sulfate and cholesterol hemisuccinate; tocopherol hemisuccinate; lipids with ether and ester-linked fatty acids; polymerized lipids (a wide variety of which are well known in the art); diacetyl phosphate; dicetyl phosphate; stearylamine; cardiolipin; phospholipids with short chain fatty acids of 6-8 carbons in length; synthetic phospholipids with asymmetric acyl chains (e.g., with one acyl chain of 6 carbons and another acyl chain of 12 carbons); ceramides; non-ionic liposomes including niosomes such as polyoxyethylene fatty acid esters, polyoxyethylene fatty alcohols, polyoxyethylene fatty alcohol ethers, polyoxyethylated sorbitan fatty acid esters, glycerol polyethylene glycol oxystearate, glycerol polyethylene glycol ricinoleate, ethoxylated soybean sterols, ethoxylated castor oil, polyoxyethylene-polyoxypropylene polymers, and polyoxyethylene fatty acid stearates; sterol aliphatic acid esters including cholesterol sulfate, cholesterol butyrate, cholesterol iso-butyrate, cholesterol palmitate, cholesterol stearate, lanosterol acetate, ergosterol palmitate, and phytosterol n-butyrate; sterol esters of sugar acids including cholesterol glucuroneide, lanosterol glucuronide, 7-dehydrocholesterol glucuronide, ergosterol glucuronide, cholesterol gluconate, lanosterol gluconate, and ergosterol gluconate; esters of sugar acids and alcohols including lauryl glucuronide, stearoyl glucuronide, myristoyl glucuronide, lauryl gluconate, myristoyl gluconate, and stearoyl gluconate; esters of sugars and aliphatic acids including sucrose laurate, fructose laurate, sucrose palmitate, sucrose stearate, glucuronic acid, gluconic acid, accharic acid, and polyuronic acid; saponins including sarsasapogenin, smilagenin, hederagenin, oleanolic acid, and digitoxigenin; glycerol dilaurate, glycerol trilaurate, glycerol dipalmitate, glycerol and glycerol esters including glycerol tripalmitate, glycerol distearate, glycerol tristearate, glycerol dimyristate, glycerol trimyristate; longchain alcohols including n-decyl alcohol, lauryl alcohol, myristyl alcohol, cetyl alcohol, and n-octadecyl alcohol; 6-(5-cholesten-3.beta.-yloxy)-l-thio-.beta.-D-galactopyranoside; digalactosyldiglyceride; 6-(5-cholesten--3.beta.-yloxy)hexyl-6-amino-6-deoxy-1-thio-.beta.-D-galacto pyranoside; 6-(5-cholesten-3.beta.-yloxy)hexyl-6-amino-6-deoxyl--1-thio-.alpha.-D-manno pyranoside; 12-(((7′-diethylaminocoumarin-3-yl)carbonyl)methylamino)-octadecanoic acid; N->>12-(((7′-diethylaminocoumarin-3-yl)carbonyl)methyl-amino) octadecanoyl!-2-aminopalmitic acid; cholesteryl)4′-trimethylammonio) butanoate; N-succinyldioleoylphosphatidylethanolamine; 1,2-dioleoyl-sn-glycerol;1,2-dipalmitoyl-sn-3-succinylglycerol; 1,3-dipalmitoyl--2-succinylglycerol;1-hexadecyl-2-palmitoyl-glycerophosphoe thanolamine and palmitoylhomocysteine, and/or combinations thereof. A particularly preferred lipid according to the presently claimed invention is a phospholipid.

A preferred therapeutically active ingredient useful in the presently claimed unilamellar vesicles is selected from the group consisting of pharaceutically active materials, labile materials, and mixtures thereof. A particularly preferred labile material is selected from the group consisting of proteins and peptides. In a preferred embodiment, the pharmaceutically active material is not subject to acidic, alkaline, enzymatic or other degradation when used in the environment of the gastrointestinal tract.

Biologically or chemically active materials which can be encapsulated by the present inventive subject matter include, but are not limited to pharmacological agents, and therapeutic agents. For example, biologically or chemically active agents suitable for use in the present invention include, but are not limited to, peptides, and particularly small peptides; hormones, and particularly hormones which by themselves do not or only a fraction of the administered dose passes through the gastro-intestinal mucosa and/or are susceptible to chemical cleavage by acids and enzymes in the gastrointestinal tract; polysaccharides, and particularly mixtures of muco-polysaccharides; carbohydrates; lipids; or any combination thereof. Further examples include, but are not limited to, human growth hormones; bovine growth hormones; growth releasing hormones; interferons; interleukin-1; insulin; heparin, and particularly low molecular weight heparin; calcitonin; erythropoietin; atrial naturetic factor; antigens; monoclonal antibodies; somatostatin; adrenocorticotropin, gonadotropin releasing hormone; oxytocin; vasopressin; cromolyn sodium (sodium or disodium chromoglycate); vancomycin; desferrioxamine (DFO); parathyroid hormone anti-microbials, including, but not limited to anti-fungal agents; or any combination thereof. In a preferred embodiment, calcitonin is the active agent.

The unilamellar vesicles of the present invention can be made by a variety of devices which provides sufficiently high shear for shear mixing. There are a large variety of these devices available on the market including a microfluidizer such as is made by Biotechnology Development Corporation, a “French”-type press, or some other device which provides a high enough shear force.

A device which is particularly useful for making the lipid vesicles of the present invention has been developed by Micro Vesicular Systems, Inc., Vineland, N.J. and is further described in U.S. Pat. No. 4,895,452.

This device has a substantially cylindrical mixing chamber with at least one tangentially located inlet orifice. One or more orifices lead to a reservoir for the lipophilic phase and at least one of the other orifices is attached to a reservoir for the aqueous phase.

The different phases are driven into the cylindrical chamber through pumps, e.g., positive displacement pumps, and intersect in such a manner as to form a turbulent flow within the chamber. The unilamellar vesicles are removed from the chamber through an axially located discharge orifice.

In the aqueous phase chamber a biologically active therapeutic is mixed with the diluent. In the lipophilic chamber the stabilizing compounds are added. Both phases are then mixed in the cylindrical chamber at about 30,000 revolutions per minute (“rpm”) while surfactants are added to the cylindrical chamber.

Several non-limiting examples of surfactants useful according to the presently claimed invention include docusate sodium, sodium lauryl sulfate, cetrimide, polyoxyethylene fatty acid esters, and sorbitan esters.

One of ordinary skill in the art without undue experimentation could vary the rpm of the high shear to produce substantially the same invention without deviating from the disclosure presented herein. Moreover, methods for the preparation of such polymer-based unilamellar vesicles will be readily apparent to those skilled in the art, in view of the present disclosure, when the present disclosure is coupled with information known in the art.

Theory of the Invention

Without limiting the theory of the invention to any particular theory, several possible explanations arise for the novel mechanisms of the transcellular drug delivery technology provided herein.

Under a Pulsed Emulsion Phenomenon Theory (“PEP”), the release of the therapeutically active material from the unilamellar vesicle is dependent on either the environmental pH or the type of ambient enzymes present. Under a pH-dependent model, the unilamellar vesicles dock to a mucosal lining and release the biologically active therapeutic when ambient pH is either neutral or non-acidic (7.0 pH>). At pH neutral sites such as the oral, pharyngeal, esophageal sites and again at the colon, the unilamellar vesicles would release the active. Highly acidic areas such as the stomach and small intestine would prevent release. Typically, release in the mouth, throat, and esophagus may be seen at about 6-8 hours after administration, while release in the colon is seen at about 12-16 hours after administration.

Under an enzyme-dependent model, a biologically present enzyme could either trigger or prevent the docking/release event. For example, protease present in the small intestine could lock-up the vesicle preventing release while lipase present in the lower GI tract could be triggering an docking/release event releasing the therapeutic into the lower intestine for absorption into the jujenum at the colon.

A Mucosal Docked Vesicle Theory posits that significant absorption only occurs at anatomical sites possessing a mucosal epithelium (i.e. epithelial tissue coated with mucous). It is possible that the unilamellar vesicle only interacts with the mucosal basal membrane or with the mucous itself. Docking/releasing events only seem to occur at mucosal surfaces. Upon a docking/releasing event, biologically active drugs sequestered in the vesicle diffuse across the mucosal basal membrane and enter the bloodstream for systemic distribution. Since the stomach and small intestine do not possess a mucosal epithelium, this would explain why no docking/release event occurs in these areas.

Another explanation for the docking/release event are VanderWaal interactions occurring between the unilamellar vesicle and the mucosal membrane. VanderWaal forces are temporary dipoles induced in one molecule by another molecule. This physical interaction would be similar to the “static cling” of plastic decals to glass used in place of adhesive decals for auto windows. VanderWaal forces may trigger docking and subsequent release.

One of ordinary skill in the art will understand that the particular theory of the invention is not limited to any single one of the above theories, or may be a combination of the above theories or involve theories as of yet not ascertainable and do not limit in any way to the ability to practice the invention as disclosed herein.

Calcitonin and human growth hormone exemplify the problems confronted in the art in designing an effective oral drug delivery system. The medicinal properties of calcitonin and human growth hormone can be readily altered using any number of techniques, but their physicochemical properties and susceptibility to enzymatic digestion have limited the design of viable delivery systems. Others among the numerous agents which are not typically amenable to oral administration are biologically active proteins such as insulin, the cytokines (e.g. interferons, IL-2, etc); erythropoietin; polysaccharides, and in particular mucopolysaccharides including, but not limited to, heparin; heparinoids; antibiotics; and other organic substances. These agents are also rapidly rendered ineffective or are destroyed in the GI tract by acid hydrolysis, enzymes, or the like.

Clinical Evaluations

An exemplified embodiment of the presently claimed invention using calcitonin for oral administration detected calcitonin blood levels at certain intervals after the dosage was given. The results show that substantial systemic absorption of calcitonin took place in the subjects typically at about 6-8 hours after administration. This indicates a release of the calcitonin in the mouth, throat, and esophagus. Further absorption of the calcitonin was also seen at about 12-16 hours after administration, which indicates absorption in the colon.

The transcellular drug delivery system of the present invention was used to prepare the following examples. All percentages are based on the percent by weight of the final delivery system or formulation prepared unless otherwise indicated and all totals equal 100% by weight.

### EXAMPLE I

| | Amount % w/w |
|---|---|
| purified water | 24.878 |
| glycerin | 48.000 |
| glacial acetic acid | 0.0225 |
| sodium acetate | 0.200 |
| sodium chloride | 0.750 |
| methylparaben | 0.090 |
| propylparaben | 0.035 |
| butylparaben | 0.024 |
| sucrose | 8.000 |
| calcitonin (Salmon) 800 unit/dose | 0.00094 |
| mineral oil | 13.000 |
| polyethylene glycol (30) | 5.00 |
| dipolyhydroxystearate | |
| TOTAL | 100.00 |

The unilamellar vesicles can be made by a variety of devices known in the art which provides sufficiently high shear for shear mixing. A device which is particularly useful has been developed by Micro Vesicular Systems, Inc., Vineland, N.J. and is further described in U.S. Pat. No. 4,895,452. Temperature utilized is dependent upon the end product desired.

The formulas described in these examples were produced by the following method:

The calcitonin and additional components of the water-soluble phase are mixed with the purified water. The ingredients of the water-insoluble external phase are mixed together in a second vessel. The water-soluble internal phase is slowly added to the water-insoluble external phase while the two phases are mixed together with a split disk stirrer until addition is complete and desired viscosity is obtained, Mixing speed is dependent upon the end product desired.

### EXAMPLE II

The method of producing Example I may be used to produce a transcellular human growth hormone delivery system according to the following formula:

| | Amount % w/w |
|---|---|
| purified water | 24.878 |
| glycerin | 48.000 |
| glacial acetic acid | 0.0225 |
| sodium acetate | 0.200 |
| sodium chloride | 0.750 |
| methylparaben | 0.090 |
| propylparaben | 0.035 |
| butylparaben | 0.024 |
| sucrose | 8.000 |
| human growth hormone 12 mg/dose | 0.00071 |
| mineral oil | 12.000 |
| polyethylene glycol (30) | 6.00 |
| dipolyhydroxystearate | |
| TOTAL | 100.00 |

The invention being thus described, it will be obvious that the same may be varied in many ways. Such variations are not to be regarded as a departure from the spirit scope of the invention and all such modifications are intended to be included within the scope of the following claims.

we claim:

1. A storage stable bioadhesive unilamellar vesicle comprising:
a) an exterior unilamellar film comprising at least one amphiphilic or hydrophobic material; and
b) an aqueous interior defined by said exterior unilamellar film, said aqueous interior comprising a therapeutically active ingredient;
wherein said vesicle is from about 100 nm to about 100 microns in size and has a neutral charge associated therewith.

2. The unilamellar vesicle of claim 1 wherein said amphiphilic or hydrophobic material is selected from the group consisting of mineral oil, lipid material, neutral fats, and mixtures thereof.

3. The unilamellar vesicle of claim 2 wherein said lipid material is a phospholipid.

4. The unilamellar vesicle of claim 1, wherein said vesicle is from about 2 microns to about 50 microns in size.

5. The unilamellar vesicle of claim 4 wherein said therapeutically active ingredient is selected from the group consisting of pharmaceutically active materials, labile materials, and mixtures thereof.

6. The unilamellar vesicle of claim 5, wherein said labile material is selected from the group consisting of proteins and peptides.

7. The unilamellar vesicle of claim 5 wherein said pharmaceutically active material is not subject to acidic, alkaline, enzymatic or degradation when used in the environment of the gastrointestinal tract.

8. A storage stable pharmaceutical composition comprising:
a) a bioadhesive unilamellar vesicle comprising:
i) an exterior unilamellar film comprising at least one amphiphilic or hydrophobic material; and
ii) an aqueous interior defined by said exterior unilamellar film, said aqueous interior comprising a therapeutically active ingredient;
wherein said vesicle is from about 100 nm to about 100 microns in size and has a neutral charge associated therewith; and
b) a pharmaceutically acceptable carrier.

9. The pharmaceutical composition of claim 8 wherein said amphiphilic or hydrophobic material is selected from the group consisting of mineral oil, lipid material, neutral fats, and mixtures thereof.

10. The pharmaceutical composition of claim 9 wherein said lipid material is a phospholipid.

11. The unilamellar vesicle of claim 8, wherein said vesicle is from about 2 microns to about 50 microns in size.

12. The pharmaceutical composition of claim 8 wherein said therapeutically active ingredient is selected from the group consisting of pharmaceutically active materials, labile materials, and mixtures thereof.

13. The pharmaceutical composition of claim 12 wherein said labile material is selected from the group consisting of proteins and peptides.

14. The pharmaceutical composition of claim 12 wherein said pharmaceutically active material is not subject to acidic, alkaline, enzymatic or degradation when used in the environment of the gastrointestinal tract.

15. A storage stable bioadhesive unilamellar vesicle comprising:
a) an exterior unilamellar film comprising at least one amphiphilic or hydrophobic material; and
b) an aqueous interior defined by said exterior unilamellar film, said aqueous interior comprising a therapeutically active ingredient;
wherein said vesicle is from about 100 nm to about 100 microns in size; and
wherein the vesicle further comprises an anionic surfactant.

16. The unilamellar vesicle of claim 15, wherein said vesicle is from about 2 microns to about 50 microns in size.

17. A bioadhesive unilamellar vesicle comprising:
a) an exterior unilamellar film comprising at least one amphiphilic or hydrophobic material; and
b) an aqueous interior defined by said exterior unilamellar film, said aqueous interior comprising water and a leukotriene;
wherein said vesicle is from about 100 nm to about 100 microns in size and has a neutral charge associated therewith.

18. The unilamellar vesicle of claim 17, wherein said vesicle is from about 2 microns to about 50 microns in size.

19. A bioadhesive unilamellar vesicle comprising:
a) an exterior unilamellar film comprising at least one amphiphilic or hydrophobic material; and
b) an aqueous interior defined by said exterior unilamellar film, said aqueous interior comprising water and a cytokine;
wherein said vesicle is from about 100 nm to about 100 microns in size and has a neutral charge associated therewith.

20. The unilamellar vesicle of claim 19, wherein said vesicle is from about 2 microns to about 50 microns in size.

21. A method of administering a storage stable labile material, which material is commonly administered as an injectable, to a patient in need thereof, comprising the step of orally, rectally, or via the colon administering to a patient comprising:
a) a bioadhesive unilamellar vesicle comprising:
i) an exterior unilamellar film comprising at least one amphiphilic or hydrophobic material; and
ii) an aqueous interior defined by said exterior unilamellar film, said aqueous interior comprising a therapeutically active ingredient;
wherein said vesicle is from about 100 nm to about 100 microns in size and has a neutral charge associated therewith; and
b) a pharmaceutically acceptable carrier.

22. A method of systemically delivering a therapeutically active ingredient to a patient in need thereof, comprising the step of administering a storage stable pharmaceutical composition to said patient, said pharmaceutical composition comprising:
a) a bioadhesive unilamellar vesicle comprising:
i) an exterior unilamellar film comprising at least one amphiphilic or hydrophobic material; and
ii) an aqueous interior defined by said exterior unilamellar film, said aqueous interior comprising a therapeutically active ingredient;
wherein said vesicle is from about 100 nm to about 100 microns in size and has a neutral charge associated therewith; and
b) a pharmaceutically acceptable carrier; and
wherein said vesicle bioadheres to the tissues of the mouth, throat, esophagus, upper gastrointestinal tract, lower gastrointestinal tract, rectum and colon.

23. The method of claim 22, wherein said amphiphilic or hydrophobic material is selected from the group consisting of mineral oil, lipid material, neutral fats, and mixtures thereof.

24. The method of claim 23, wherein said lipid material is a phospholipid.

25. The method of claim 22, wherein said unilamellar vesicle is from about 2 microns to about 50 microns in size.

26. The method of claim 22, wherein said therapeutically active ingredient is selected from the group consisting of pharmaceutically active materials, labile materials, and mixtures thereof.

27. The method of claim 26 wherein said labile material is selected from the group consisting of proteins and peptides.

28. The method of claim 26, wherein said pharmaceutically active material is not subject to acidic, alkaline, enzymatic, or degradation when used in the environment of the gastrointestinal tract.

29. A method of systemically delivering a pharmaceutically active ingredient to a patient in need thereof, comprising the step of administering a storage stable pharmaceutical composition to said patient, said pharmaceutical composition comprising:
a) a bioadhesive unilamellar vesicle comprising:
i) an exterior unilamellar film comprising at least one amphiphilic or hydrophobic material; and
ii) an aqueous interior defined by said exterior unilamellar film, said aqueous interior comprising a therapeutically active labile ingredient;
wherein said vesicle is from about 100 nm to about 100 microns in size and has a neutral charge associated therewith; and
b) a pharmaceutically acceptable carrier;
wherein said pharmaceutical composition is administered orally or rectally.

30. The method of claim 29, wherein said amphiphilic or hydrophobic material is selected from the group consisting of mineral oil, lipid material, neutral fats, and mixtures thereof.

31. The method of claim 30 wherein said lipid material is a phospholipid.

32. The method of claim 29, wherein said unilamellar vesicle is from about 2 microns to about 50 microns in size.

33. The method of claim 29, wherein said therapeutically active ingredient is selected from the group consisting of pharmaceutically active materials, labile materials, and mixtures thereof.

34. The method of claim 33, wherein said labile material is selected from the group consisting of proteins and peptides.

35. The method of claim 33, wherein said pharmaceutically active material is not subject to acidic, alkaline, enzymatic, or other degradation when used in the environment of the gastrointestinal tract.
